(19) United States
(12) Patent Application Publication
(10) Pub. No.: US 2020/0126446 A1
(43) Pub. Date: Apr. 23, 2020

(54) Gait Teaching System and Gait Teaching Method

(71) Applicant: NO NEW FOLK STUDIO INC., Tokyo (JP)

(72) Inventors: Yuya KIKUKAWA, Tokyo (JP); Takaharu KANAI, Tokyo (JP)

(73) Assignee: No New Folk Studio Inc., Tokyo (JP)

(21) Appl. No.: 16/490,881

(22) PCT Filed: Mar. 2, 2018
(86) PCT No.: PCT/JP2018/008181

§ 371 Date: Sep. 3, 2019

(30) Foreign Application Priority Data
Mar. 3, 2017 (JP) 2017-040294

(51) Int. Cl. G09B 19/00; G09B 5/00; A61B 5/11; A61B 5/00; A43B 3/00

(52) U.S. Cl. CPC G09B 19/003; G09B 5/00; G01P 15/18; A61B 5/6802; A43B 3/0005; A61B 5/11

(57) ABSTRACT

In a walking teaching system including footwear and an external device, the footwear includes a sensor unit configured to detect a motion of the footwear, and a first transmission unit configured to transmit detection information detected by the sensor unit to the external device, and the external device includes a first reception unit configured to receive the detection information, a first storage unit configured to store model information serving as a model of walking, and a presentation unit configured to present a way to walk to a user wearing the footwear on the basis of the detection information and the model information.

### TECHNICAL FIELD

The present invention relates to a walking teaching system and a walking teaching method for teaching a user wearing footwear a way to walk.

### BACKGROUND ART

In the related art, there is a running support wearable terminal that performs support according to a state of a user when the user is running, as in a marathon (for example, Patent Literature 1). This running support wearable terminal holds reference information serving as a reference for a running state of the user, compares a reference state with a running state, and performs a notification when running is different from the reference.

### CITATION LIST

### Patent Literature

### [Patent Literature 1]

Japanese Unexamined Patent Application Publication No. 2015-206601

### SUMMARY OF INVENTION

### Technical Problem

Incidentally, in the technology described in Patent Literature 1 above, a better way to run cannot be proposed to a user. Further, although a person performs whichever of walking and running is more natural for the person, in some cases walking or running may become burdensome to a person and it may go unnoticed. In particular, in the case of a woman, such a tendency becomes notable when the woman wears shoes such as pumps having high heels. When a user walks with such a burden, sometimes, there is a problem in that health of the user is likely to be damaged.

Therefore, the present invention has been made in view of the above problems, and an object thereof is to provide a walking teaching system capable of presenting a more appropriate way to walk to a user.

### Solution to Problem

In order to solve the above problems, a walking teaching system according to an aspect of the present invention is a walking teaching system including footwear and an external device, wherein the footwear includes a sensor unit configured to detect a motion of the footwear; and a first transmission unit configured to transmit detection information detected by the sensor unit to the external device, and the external device includes a first reception unit configured to receive the detection information; a first storage unit configured to store model information serving as a model of walking; and a presentation unit configured to present a way to walk to a user wearing the footwear on the basis of the detection information and the model information.

In order to solve the above-described problem, a walking teaching method according to an aspect of the present invention is a walking teaching method using a walking teaching system including footwear and an external device, the walking teaching method including: a detection step of detecting a motion of the footwear by the footwear; a transmission step of transmitting, by the footwear, detection information detected in the detection step to the external device; a storage step of storing, by the external device, model information serving as a model of walking; and a presentation step of presenting, by the external device, a way to walk to a user wearing the footwear on the basis of the detection information and the model information.

The walking teaching system may further include a management device, the external device may further include a second transmission unit configured to transmit the received detection information to the management device, the management device may include a second reception unit configured to receive the detection information; a second storage unit configured to store the received detection information; a generation unit configured to generate correction information for correcting the model information on the basis of a plurality of pieces of detection information stored in the storage unit; and a third transmission unit configured to transmit the correction information to the management device, the first reception unit may further receive the correction information, the external device may further include a correction unit configured to correct the model information on the basis of the correction information, and the presentation unit may present the way to walk on the basis of the model information corrected by the correction unit.

In the walking teaching system, the first storage unit may further store user information indicating a feature of a user using the footwear, the first storage unit may store a plurality of pieces of model information associated with the feature of the user, and the presentation unit may present the way to walk on the basis of the model information corresponding to the feature of the user indicated by the user information and the detection information.

In the walking teaching system, the second transmission unit may further transmit the user information to the management device, the second reception unit may receive the user information, and the generation unit may generate correction information according to the feature of the user indicated by the user information.

In the walking teaching system, the external device may further include a second generation unit configured to generate a control signal related to control of output of at least one of light, sound, and vibration on the basis of the detection information, the second transmission unit may further transmit the control signal to the footwear, and the footwear further may include a third reception unit configured to receive the control signal; and an output unit configured to perform output of at least one of light, sound, and vibration on the basis of the control signal.

### Advantageous Effects of Invention

The walking teaching device according to an aspect of the present invention can perform teaching regarding a way to walk to a user on the basis of the motion of footwear that is detected according to a motion of a user.

### BRIEF DESCRIPTION OF DRAWINGS

FIG. 1 is a schematic diagram illustrating an overview of a walking teaching system.

FIG. 2 is a block diagram illustrating an example of a configuration of the walking teaching system and a functional configuration of each device constituting the system.

FIG. 3(a) is an appearance view illustrating an external appearance of footwear, and FIG. 3(b) is an appearance view illustrating a configuration of a tongue portion.

FIG. 4(a) is a plan view of a sole portion, FIG. 4(b) is a cross-sectional view of the sole portion, and FIG. 4(c) is a cross-sectional view of the sole portion, which is a cross-sectional view illustrating an example in which an output unit is disposed.

FIG. 5(a) is a perspective view of the sole portion, and FIG. 5(b) is a perspective view of the sole portion, which is a view illustrating a state in which an output unit 130 is disposed.

FIG. 6 is a flowchart illustrating an operation of the external device 200.

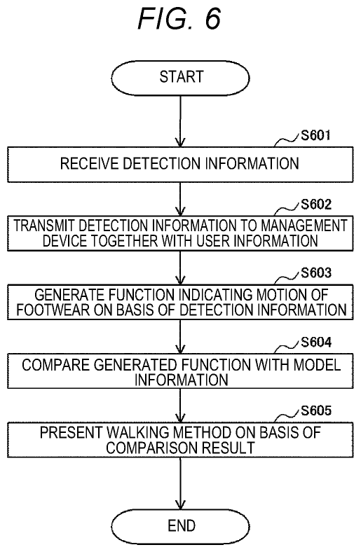

FIG. 7 is a flowchart illustrating an operation of the external device 200 when correction information is received.

FIG. 8 is a block diagram illustrating an example of a configuration of a walking teaching system and another configuration of each device constituting the system.

### DESCRIPTION OF EMBODIMENTS

Hereinafter, a server according to an embodiment of the present invention will be described in detail with reference to the drawings.

### Embodiment

<Configuration>

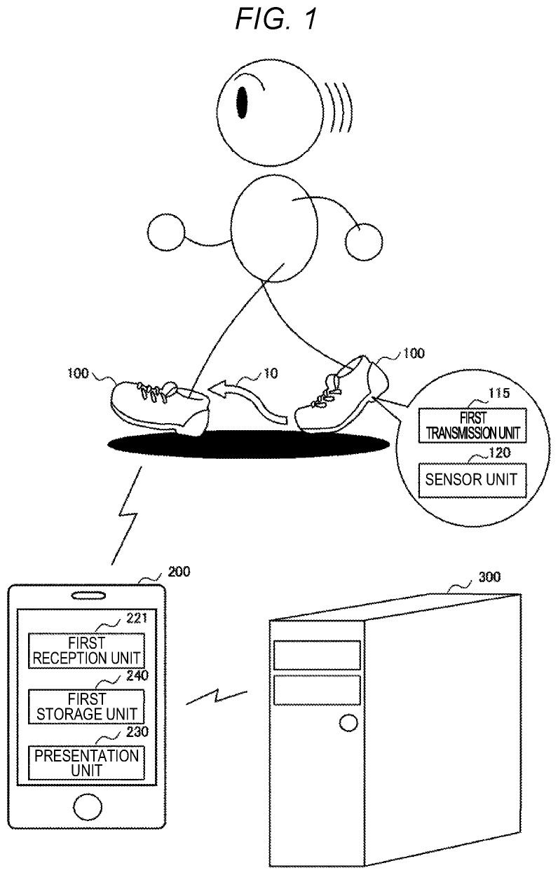

FIG. 1 is a schematic diagram illustrating an overview of a walking teaching system. As illustrated in FIG. 1, the walking teaching system includes footwear 100 and an external device.

The footwear 100 includes a sensor unit 120 and a transmission unit 115 (a first transmission unit). The footwear 100 is worn on feet of a user, and is, for example, sneakers, leather shoes, pumps, or sandals. The footwear 100 has a space for placing at least the sensor unit 120 and the transmission unit 115.

The sensor unit 120 is a sensor having a function of detecting a motion of the footwear 100 when the user wears the footwear 100 and moves. The motion of the footwear 100 can be detected as a motion of the foot of the user. The sensor unit 120 can be realized from, for example, an acceleration sensor that detects an acceleration of three axes (for example, two axes perpendicular to each other in a horizontal surface and an axis perpendicular to the two axes), and an angular velocity sensor for detecting rotation angles of the three axes. The sensor unit 120 may further include a geomagnetic sensor that detects geomagnetism in directions of the three axes, and function as a nine-axis sensor. Hereinafter, sensing data detected by the sensor unit 120 is referred to as detection information. The detection information includes at least acceleration information of the three axes and angular velocity information of the three axes.

The transmission unit 115 transmits the detection information detected by the sensor unit 120 to an external device 200. The transmission unit 115 can be realized by a communication interface, for example.

The external device 200 is, for example, a mobile terminal held by the user, and can be realized by, for example, a smartphone, a mobile phone, a tablet terminal, or a laptop PC.

The external device 200 includes a first reception unit 221, a first storage unit 240, and a presentation unit 230.

The first reception unit 221 receives sensing data transmitted from the footwear 100. The first reception unit 221 can be realized by a communication interface, for example.

The first storage unit 240 stores model information serving as a model for walking. The first storage unit 240 can be realized by various small recording media such as a flash memory, for example. The model information is information that serves as a model for walking for the user, and indicates, for example, a temporal change in the detection information measured when the ideally walking user wears the footwear 100. The model information may be information indicating motion for one step or may be information indicating motion for several steps.

The presentation unit 230 presents a way to walk to the user wearing the footwear 100 on the basis of the received detection information and the model information stored in the first storage unit 240.

The external device 200 may transmit the received detection information indicating the motion of the user at the time of walking or running to a management device 300. The management device 300 may be a computer device that collects and manages the detection information indicating the motion of the user at the time of walking. Accordingly, the management device 300 can function as a server that can handle the detection information of various methods of walking of users as big data.

The walking teaching system described above will be described in more detail.

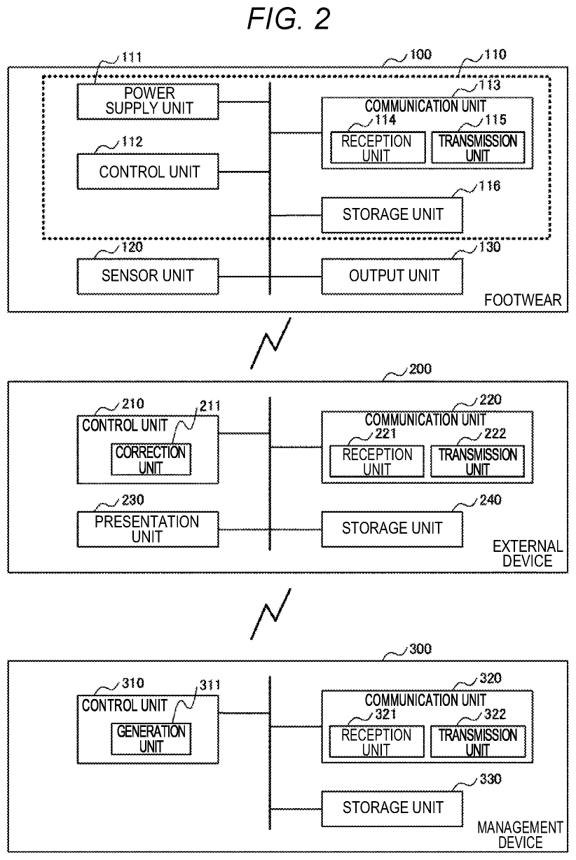

FIG. 2 is a system diagram illustrating a configuration of the walking teaching system, and is a block diagram illustrating a functional configuration of each device constituting the walking teaching system.

As illustrated in FIG. 2, the footwear 100 performs communication with the external device 200. Further, the external device 200 executes communication with the management device 300. Although not illustrated, the management device 300 executes communication with the external devices 200 of a large number of different users to collect the detection information from each external device 200.

As illustrated in FIG. 2, the footwear 100 includes a module 110, the sensor unit 120, and an output unit 130. The module 110, the sensor unit 120, and the output unit 130 are connected via a bus. The connection may be any of wired and wireless connection as long as information can be transferred between them. The sensor unit 120 and the output unit 130 may be configured to be connected via the module 110.

The module 110 includes a power supply unit 111, a control unit 112, and a communication unit 113.

The power supply unit 111 is a battery having a function of supplying driving power to each unit constituting the footwear 100.

The control unit 112 is a processor having a function of controlling each unit of the footwear 100. The control unit 112 controls each unit of the footwear 100 by executing a control program stored in a storage unit 116. The control unit 112 requests the communication unit 113 to transmit the detection information transferred from the sensor unit 120 to the external device 200. Further, the control unit 112 causes the output unit 130 to generate light according to a control signal received by the communication unit 113.

The communication unit 113 is a communication interface having a function of executing communication with the external device 200. The communication unit 113 includes a reception unit 114 and a transmission unit 115 (a first transmission unit). The communication unit 113 communicates with the external device 200 through wireless communication. The communication unit 113 may perform communication using any communication standard as long as the communication unit 113 can communicate with the external device 200. As a communication standard used by the communication unit 113, for example, a standard such as Bluetooth Low Energy (registered trademark), Bluetooth (registered trademark), 3rd Generation (3G), 4th Generation (4G), or Long Term Evolution (LTE) may be used. As a communication standard used by the communication unit 113, it is preferable to use a communication standard in which a power consumption amount necessary for communication is small when communication with the external device 200 is performed.

The reception unit 114 receives a control signal for controlling the output unit 130 from the external device 200. The reception unit 114 transmits the received control signal to the control unit 112.

The transmission unit 115 transmits the detection information indicating the motion of the footwear 100 detected by the sensor unit 120 to the external device 200.

The sensor unit 120 detects a motion that is detected when the user wearing the footwear 100 moves. As described above, the sensor unit 120 detects at least the acceleration in the directions of the three axes and the angular velocity information indicating rotation angles of the three axes, and transfers detected information to the control unit 112 as the detection information.

The output unit 130 performs output based on a control signal received by the reception unit 114 of the communication unit 113 according to an instruction from the control unit 112. Here, the output unit 130 emits light, and is realized by an LED, for example. The LED can emit a plurality of colors. The LED emits light in emission color and an emission pattern indicated by the control signal. The output of the output unit 130 is emission of light for notifying the user walking with the footwear 100 that his or her way to walk is different from the model when his or her way to walk is different from the model. The light emission of the output unit 130 may simply be light emission according to music that is output from the external device 200 or light emission according to the motion of the footwear 100.

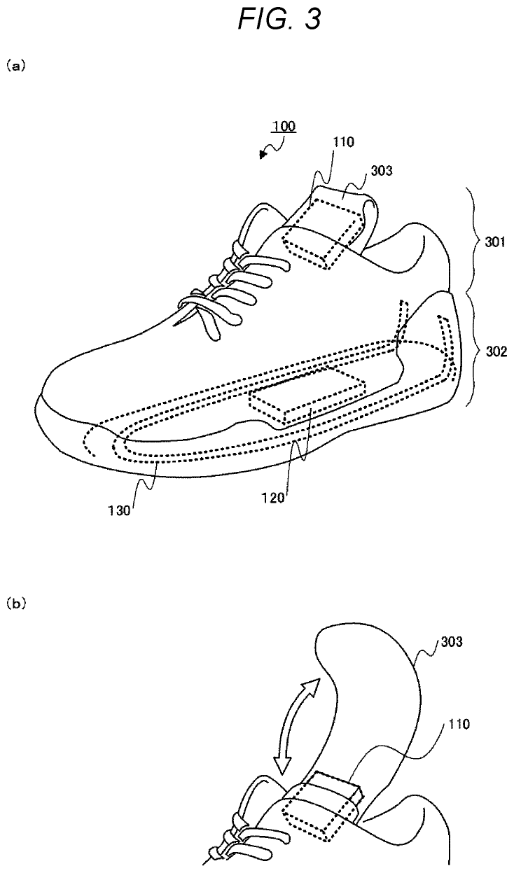

Here, an example of an appearance of the footwear 100 and a disposition of each unit will be described. FIG. 3 is a diagram illustrating an example of the footwear 100, and is a diagram illustrating an example of an appearance of the footwear 100 and a disposition of each unit inside the footwear 100.

FIG. 3(a) is an appearance view illustrating a configuration of the footwear 100. As illustrated in FIG. 3(a), the footwear 100 includes an upper surface side of the footwear 100, which is an upper portion 301 that covers and fixes an instep of the user wearing the footwear 100, and a bottom surface side of the footwear 100, which is a sole portion 302 having a function of absorbing an impact. A tongue portion 303 for protecting the instep of the user is provided in the upper portion 301. The module 110 is provided in the tongue portion 303. As illustrated in FIG. 3(b), the module 110 inserted into a pocket provided in the tongue portion 303 can be exposed by opening the tongue portion 303. Although not illustrated, the module 110 has a terminal (for example, a USB terminal) for receiving power supply. By opening the tongue portion 303 as illustrated in FIG. 3(b), the terminal is connected to an external power source, the power supply is received, and electricity is accumulated in the power supply unit 111.

In the footwear 100, the sole portion 302 includes the output unit 130 and the sensor unit 120, as described above. The sensor unit 120 is provided inside the sole portion 302, which is inside a shank portion at a position corresponding to an arch of the foot of the user. Although not illustrated, the sensor unit 120 is connected to the module 110 through the inside of the footwear 100, operates with power supplied from the power supply unit 111 inside the module 110, and transfers sensor data (detection information) to the module 110. Accordingly, the detection information detected by the sensor unit 120 is transferred to the external device 200 by the communication unit 113.

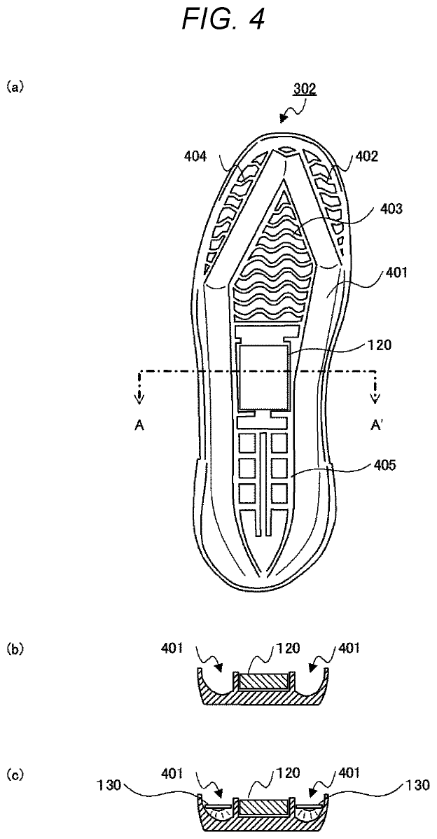

FIG. 4(a) is a plan view of the sole portion 302, and FIG. 4(b) is a cross-sectional view of the sole portion 302 of FIG. 4(a) taken along a line A-A′. As illustrated in FIG. 4(a), the sole portion 302 includes a groove portion 401 for placing the output unit 130. The groove portion 401 is provided inside the sole portion 302, which is in an outer peripheral portion of the sole portion 302, so that the groove portion 401 is along an outer edge of the sole portion 302. The groove portion 401 is recessed for the output unit 130 to be placed therein, and an LED tape is provided in the groove portion 401 as the output unit 130. As illustrated in FIG. 4(a), the sensor unit 120 is provided at a location at which the groove portion 401 is not provided, which is at a position facing the arch of the foot of the user inside the sole portion 302. The location is a position referred to as a so-called shank portion in a structure of the footwear 100. In the sole portion 302, impact absorbing ribs 402 to 405 are provided at positions at which the groove portion 401 and the sensor unit 120 are not provided. The ribs 402 and 403 are provided on the toe side of the user of the sole portion 302, which is on an outer peripheral side relative to the groove portion 401. Accordingly, it is possible to absorb an impact on a front end portion of the footwear 100 with respect to the footwear 100 to reduce a likelihood of failure of the output unit 130 provided in the groove portion 401 and reduce a burden on the foot of the user. Similarly, ribs 404 and 405 are located at a center of the footwear 100 and absorb an impact on the footwear to reduce a likelihood of failure of the output unit 130 provided in the groove portion 401 and reduce a burden on the foot of the user.

FIG. 4(c) is a cross-sectional view of the sole portion 302 and illustrates a state in which the LED tape is placed as the output unit 130. As illustrated in FIG. 4(c), the output unit 130 is placed with a light emitting surface directed to a bottom surface side of the footwear 100. That is, the bottom surface of the footwear 100 emits light. The inventors have found that when the LED tape is installed along a side surface side of the sole portion 302 so that light is emitted from the side surface, a breakage rate of the LED tape increases, and particularly, a bending rate of a toe portion increases and the breakage rate increases. Therefore, a configuration in which the LED tape is placed with a light emitting surface of the LED tape directed to the bottom surface side of the sole portion 302 as illustrated in FIG. 4(c) has been conceived as a result of searching for a way to place the LED tape so that the breakage rate is further reduced. Since the sole portion 302 is configured of a transparent or translucent resin having high impact absorption, it is possible to provide the footwear 100 in which light emitted from the LED tape is transmitted, and as a result, light is emitted from the bottom surface of the sole portion 302.

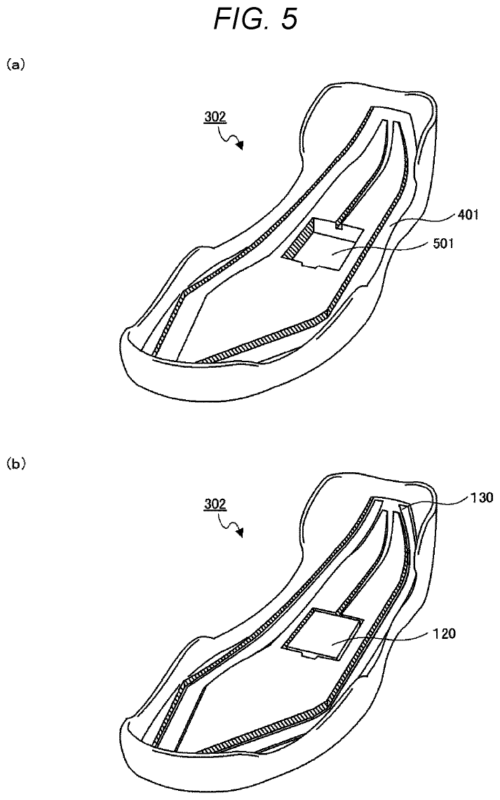

FIG. 5 is a perspective view of the sole portion 302 provided to make it easier to understand a structure of the sole portion 302. FIG. 5(a) is a perspective view illustrating a state in which the sensor unit 120 and the output unit 130 are not placed in the sole portion 302, and FIG. 5(b) is a perspective view illustrating a state in which the output unit 106 and the sensor unit 120 are placed in the sole portion 302. As can be understood from a comparison between FIG. 5(a) and FIG. 5(b), the output unit 130 that is an LED tape is placed in the groove portion 401 and provided on an outer peripheral portion of a bottom surface of the sole portion 302. Further, the sensor unit 120 is provided in a dent 501 provided in the sole portion 302. The dent 501 is configured to substantially match an outer diameter of the sensor unit 120. Accordingly, it is possible to prevent rattling as much as possible when the sensor unit 120 is placed in the dent 501, and to detect a pure motion of the footwear 100 with respect to the detection of the motion using the sensor unit 120. It is preferable for the sensor unit 120 to be provided in the sole portion 302 in order to improve a system for detecting the motion of the footwear 100.

Referring back to FIG. 2, the description will return to the external device 200 and the management device 300.

As illustrated in FIG. 2, the external device 200 includes a control unit 210, a communication unit 220, a presentation unit 230, and a storage unit 240.

The control unit 210 is a processor having a function of controlling each unit of the external device 200. The control unit 210 controls each unit of the external device 200 by executing a control program stored in the storage unit 240. The control unit 210 determines whether or not a walking operation of the user walking with the footwear 100 is ideal on the basis of the received detection information and the model information stored in the storage unit 240. The determination is performed, for example, on the basis of a magnitude of a correlation value obtained by correlating the model information with the detection information. That is, the control unit 210 determines that the walking operation is closer to the model (closer to an ideal way to walk) when the correlation value is greater, and that the walking operation is farther from the model (farther from the ideal way to walk) when the correlation value is smaller. The control unit 210 generates a signal by which a light emission intensity output by the output unit 130 is increased in order to alert the user when the correlation value is smaller, and generates a signal by which the light emission intensity is decreased when the correlation value is greater. Further, the control unit 210 generates presentation information indicating how the user is to walk on the basis of a difference between the model information and the detection information, and transfers the presentation information to the presentation unit 230.

Further, the control unit 210 includes a correction unit 211. The correction unit 211 corrects the model information according to the correction information received from the management device 300. The correction is performed, for example, by changing a numerical value of the acceleration value at a certain timing in the correction information the correction information or changing a numerical value of the angular velocity with the correction information.

The communication unit 220 is a communication interface having a function of executing communication with the footwear 100 and the management device 300. The communication unit 220 includes a reception unit 221 and a transmission unit 222. The communication unit 220 communicates with the footwear 100 through wireless communication. Further, the communication unit 220 communicates with the management device 300 through wired communication or wireless communication. The communication unit 220 may perform communication according to any communication standard as long as the communication unit 220 can communicate with the footwear 100 and the management device 300. For example, the communication unit 220 performs communication according to a communication standard such as Ethernet (registered trademark) or Bluetooth Low Energy.

The presentation unit 230 makes a proposal regarding the user the way to walk who uses the footwear 100 on the basis of the presentation information transferred from the control unit 210. The proposal can be performed through a display of text or a picture, voice notification, or the like. The presentation unit 230 can be realized by, for example, a display device provided in the external device 200, or a speaker. When the presentation unit 230 is the display device, the presentation unit 230 may teach the user wearing the footwear 100 a way to walk with a sentence or a picture that teaches the way to walk. Alternatively, when the presentation unit 230 is the speaker, the presentation unit 230 may teach the user the way to walk wearing the footwear 100 by voice.

The presentation unit 230, for example, presents that a right foot of the user is to be further raised when it is analyzed that the right foot of the user is insufficiently raised as a result of a comparison between the detection information and the model information in the control unit 210, presents that any one of grounding times is to be shortened (or lengthened) in a direction in which a deviation of the grounding time is corrected when a grounding time interval between left and right feet are different, or presents a grounding location or a location at which the foot is separated from a ground surface when the grounding location or the location at which the foot is separated from the ground surface is not appropriate between a time when the foot is grounded and a time when the foot is separated from the ground surface at the time of walking.

The storage unit 240 is a recording medium having a function of storing various programs and data necessary for an operation of the external device 200. The storage unit 240 can be realized by, for example, an HDD, an SSD, or a flash memory. The storage unit 240 stores model information serving as a model for walking. The model information is, for example, information indicating a temporal change in the motion of the footwear detected by the sensor unit 120 when a user ideally walking walks with the footwear 100. The model information may be information indicating a motion for one step or may be information indicating a motion for a plurality of steps.

Next, the management device 300 will be described. As illustrated in FIG. 2, the management device 300 includes a control unit 310, a communication unit 320, and a storage unit 330.

The control unit 310 is a processor having a function of controlling each unit of the management device 300. The control unit 310 controls each unit of the management device 300 by executing a control program stored in the storage unit 330. The control unit 310 includes a generation unit 311.

The generation unit 311 generates correction information for correcting the model information stored in the external device 200. The correction information is generated as follows, for example. The generation unit 311 selects a predetermined number or more of pieces of motion information having a predetermined correlation value or more with respect to the model information from among the motion information of the footwear 100 of a plurality of users at the time of walking, which is stored in the storage unit 330. An average of the selected detection information is obtained and a difference value between the average and the model information is calculated. The generation unit 311 uses the difference value as the correction information. A trigger for generation of the correction information in the generation unit 311 may be any trigger, may be that a manager of the management device 300 inputs a generation instruction, or may be that a certain number or more of pieces of detection information are collected.

The communication unit 320 is a communication interface having a function of executing communication with a plurality of external devices 200. The communication unit 320 communicates with the external device 200 through wired communication or wireless communication. The communication unit 320 may perform communication according to any communication standard as long as the communication unit 320 can communicate with the external device 200. For example, the communication unit 320 performs communication according to a communication standard such as Ethernet (registered trademark) or Bluetooth. The communication unit 320 includes a reception unit 321 and a transmission unit 322.

The storage unit 330 is a recording medium having a function of storing various programs and data necessary for an operation of the management device 300. The storage unit 330 can be realized by, for example, an HDD, an SSD, or a flash memory. The storage unit 330 stores and manages the detection information indicating a motion of walking of the user collected by each external device 200 in association with each user. Further, the storage unit 330 also stores the model information stored in the external device 200.

The above is the description of each device related to the walking teaching system.

<Operation>

An operation of the external device 200 that teaches the user a way to walk in the walking teaching system will be described herein.

The reception unit 221 of the external device 200 receives the detection information obtained through sensing of the sensor unit 120 from the transmission unit 115 of the footwear 100 (step S601).

The control unit 210 causes the transmission unit 222 to transmit the received detection information to the management device 300 together with information on the user who is using the footwear 100 (step S602). The transmission is not an essential component in this flow and may be performed at a different timing.

The control unit 210 generates a function indicating a temporal change in the motion of the footwear on the basis of the received detection information (step S603).

The control unit 210 correlates the function generated on the basis of the detection information with the model information and performs comparison (step S604).

The control unit 210 generates presentation information for presenting the way to walk on the basis of a correlation result. Further, the control unit 210 generates a control signal for causing the output unit 130 of the footwear 100 to perform an output related to the way to walk, and causes the transmission unit 222 to transmit the control signal to the footwear 100.

The presentation unit 230 performs presentation regarding the way to walk (text information, chart, voice, or the like) according to the transferred presentation information (step S605).

Accordingly, the walking teaching system can perform teaching regarding walking to the user. Further, since the external device 200 transmits a control signal for causing an output regarding the way to walk to be performed to the footwear 100 and the output unit 130 performs the output according to the control signal, it is possible to cause the user to recognize that the presentation regarding walking is being performed in the external device 200. Further, it is also possible to cause the user to recognize that the way to walk is incorrect.

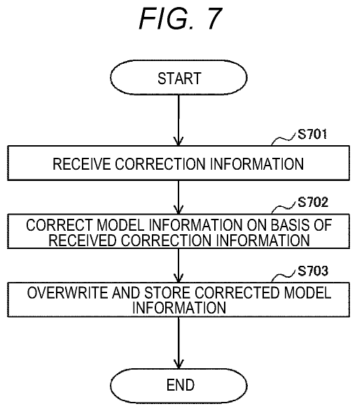

FIG. 7 is a flowchart illustrating an operation when the external device 200 receives the correction information. As illustrated in FIG. 7, the reception unit 221 of the external device 200 receives correction information for correcting the model information from the management device 300 (step S701). The reception unit 221 transfers the received correction information to the control unit 210.

The control unit 210 reads the model information stored in the storage unit 240 when the correction information is transferred. The correction unit 211 corrects the read model information with the transferred correction information (step S702).

The control unit 210 overwrites and stores the corrected model information in the storage unit 240 (step S703).

Accordingly, the model information stored in the external device 200 is appropriately updated, such that the footwear 100 can receive teaching regarding walking according to the latest model information from time to time. A function of a pacemaker such as speed increase and decrease is performed in a technology illustrated in Patent Literature 1 above, whereas with the walking teaching system including the footwear 100, the present invention is not limited to such speed, and various teachings regarding walking (a posture, a way of putting a foot on the ground, a way of separating a foot from the ground, a way of raising a foot, a way of lowering a foot, rhythm, . . . ) can be performed. The walking teaching system can contribute to promotion of health of the user by teaching the user a correct way to walk.

<Supplement>

It goes without saying that the device according to the above embodiment is not limited to the above embodiment, and may be realized by other schemes. Hereinafter, various modification examples will be described.

(1) In the above embodiment, the output unit 130 outputs the light, but the present invention is not limited thereto. The output unit 130 may be, for example, a speaker and output a sound. The control signal in this case may be generated on the basis of detection information or information on a sound stored in the external device 200 or a sound collected with a microphone (not illustrated) or the like by the external device. For example, when the walking of the user is not correct, the sound may be a beep sound for notifying that the walking of the user is not correct or may be music having the same meaning. Further, a configuration in which a loud sound is output as the detection information deviates from the model information in this case may be adopted.

Further, the output unit 130 may output vibration with a vibrator (vibration motor), for example. The control signal in this case may notify that the walking of the user is not correct by vibration when the walking of the user is not correct. As a vibration way in this case, for example, the vibration occurs violently according to a degree of deviation between the detection information obtained by detecting the motion of the footwear 100 and the model information as the deviation from the model increases.

Further, the output unit 130 may be configured to output any of light, sound, and vibration or may be configured to output two or more of the light, sound, and vibration.

(2) Although the external device 200 generates a control signal for controlling the output from the output unit 130 in the above embodiment, the control unit 112 of the footwear 100 may generate the control signal and control the output unit 130.

(3) In the above embodiment, the module 110 is provided in the tongue portion 303 of the footwear 100, but the present invention is not limited thereto. The module 110 or some of the respective functional units constituting the module 110 may be provided at a position at which the sensor unit 120 is provided.

(4) In the above embodiment, shoes such as sneakers are shown as an example of the footwear 100, but the present invention is not limited thereto. The footwear 100 may be any footwear as long as the user wears the footwear on the foot and the footwear has a space in which the module 110 or the sensor unit 120 is included. For example, an example of the footwear 100 may include pumps or high heels for a woman. In these cases, it is conceivable that the module 110, the sensor unit 120, the output unit 130, or the like is provided inside a heel member. With the footwear according to the present invention, it is possible to prevent or suppress a health damage caused by an incorrect way to walk by teaching an ideal way to walk, particularly, when a woman wears shoes such as stilettos and walks.

(5) In the above embodiment, the upper portion 301 and the sole portion 302 of the footwear 100 may be configured to be detachable from each other. In the case of the pumps for a woman, the upper portion and the heel portion may be configured to be detachable from each other. With such a configuration, fashionability of the footwear 100 can be improved. Further, when the output unit 130 is an LED tape, a probability of breakage of the LED tape due to expansion and contraction of the footwear 100 when the footwear 100 is used tends to be higher than that of other members, but maintenance of the LED tape is facilitated. Further, a configuration for detachability can be realized, for example, by a fastener, a hook-and-loop fastener, a screw, a binding, fitting of an upper portion constituting a concave portion or a protrusion and a bottom portion (a sole portion or a heel portion) constituting a protrusion or a concave portion, or the like. In this case, when the respective functional units constituting the footwear 100 are separately disposed in the upper portion and the bottom portion, it is necessary to adopt a configuration so that the functional units can communicate with each other. For example, a communication function may also be provided at the bottom portion so that wireless communication with the module provided at the upper portion can be executed, or a connection terminal may be provided at a predetermined position of the upper portion, which is a position in contact with the bottom portion, and the same connection terminal may also be provided in the bottom portion so that communication may be executed through wired communication through contact of these connection terminals.

(6) In the above embodiment, the model information may be stored according to a feature of the user. The feature of the user includes information such as a body shape, sex, and age of the user. This is because an ideal way to walk is likely to vary depending on the body shape, sex, and age of the user.

Therefore, the management device 300 may hold the model information corresponding to the feature of the user. The external device 200 may receive an input of information indicating the feature of the user using the footwear 100, and transmit the information to the management device 300. Thus, the model information according to the feature of the user may be acquired. Accordingly, it is possible to teach the way to walk suitable for each user.

Similarly, the correction information may be corrected according to the feature of the user. For example, for a user whose weight is equal to or greater than a predetermined weight, model information in which the user walks in a way to walk in which it is difficult for a burden to be applied on a knee of the user may be used, and the correction information may be correction information for correction such that it is further difficult for a burden to be applied on a knee of the user.

(7) In the above embodiment, the correction unit 211 of the external device 200 corrects the model information on the basis of the correction information generated by the generation unit 311 of the management device 300, and overwrites and stores the model information, but the present invention is not limited thereto. The generation unit 311 of the management device 300 may be configured to generate new model information itself and transmit the new model information to the external device 200, and the external device 200 may be configured to overwrite and store the received model information.

(8) Although the 9-axis sensor is used as an example of the sensor unit 120 in the above embodiment, other sensors may be used as long as user information can be acquired. For example, when pressure sensors are included, a weight of the user can be measured with a sum of measured values of the pressure sensors of both feet. Further, with the pressure sensors, it is possible to specify a change in load on the foot of the user. Alternatively, a temperature sensor may be included inside the footwear 100 to measure an approximate body temperature of the user, or a humidity sensor may be included to measure a humidity inside the footwear 100 and obtain a discomfort index for the user inside the footwear 100 from the humidity. Information obtained from such various sensors may be sent to the external device 200 or the management device 300 and may be useful for health management of the user. For example, an average body temperature of the user is calculated from the body temperature obtained from the temperature sensor, and when the body temperature measured at a certain time is higher than the average body temperature by a certain value or more, a UI for inquiring the user about whether there is no fever is displayed on the external device 200 such that the UI can be useful for health management of the user. Alternatively, a transition of the weight calculated using the pressure sensor may be managed, for example, a degree of increase in weight in a predetermined period of time may be calculated, and when the degree of increase exceeds a predetermined percentage, the external device 200 may teach keeping temperance in mind. Alternatively, when the discomfort index calculated on the basis of sensing of the humidity sensor or the temperature sensor exceeds a predetermined threshold value, teaching may be performed to propose taking off the footwear 200. Thus, teaching of things other than walking can be performed depending on a type of sensor included in the footwear 100.

(9) In the above embodiment, the external device 200 performs walking teaching on the basis of the model information and the sensing data from the sensor unit 120 of the footwear 100, but this is an example. It goes without saying that walking teaching may be performed using other schemes. For example, a function f(x) for walking teaching using the sensing data from the sensor unit 120 as an input may be stored with respect to a walking trajectory in advance, and the walking teaching may be performed on the basis of an output value from the function f(x).

For example, when rhythm of the walking of the user is specified from a periodicity specified from the sensing data and the rhythm is not constant or a rhythm balance is different between the left and right feet, the teaching can be performed so that the user walks with a constant rhythm or the teaching can be performed so that a stride length of one side is adjusted such that the left and right rhythms are balanced. Alternatively, with the walking teaching system, an inclination of the foot can be specified from the sensing data, and therefore, when the way the user puts his or her feet on the ground is specified, for example, when the user walking with the entire sole of the foot put on the ground can be specified, teaching may be performed so that the user puts his or her feet down from the heels. Alternatively, with the walking teaching system, when the user is shuffling, this can be specified from the sensing data, and thus the user can be taught to walk with raised feet. Alternatively, with the walking teaching system, an inclination with respect to the left and right in an advancing direction of the foot can also be specified, and therefore, for example, when leaning to the outside of the arch of the foot of a predetermined value or more at the time of walking of the user can be detected, a direction thereof may be taught and application of a pressure to only a part of the foot may be suppressed. Further, more complex teaching can also be performed in combination with other sensors, as illustrated in the supplement (8). For example, a load on the footwear 100 and a transition thereof can be measured by using a pressure sensor, in addition to the 9-axis sensor, as the sensor unit 120. Therefore, for example, it may be found that the user puts his or her foot on the ground too vigorously, and the user may be taught to put his or her foot on the ground slowly. Thus, the walking teaching system can perform various teachings regarding walking.

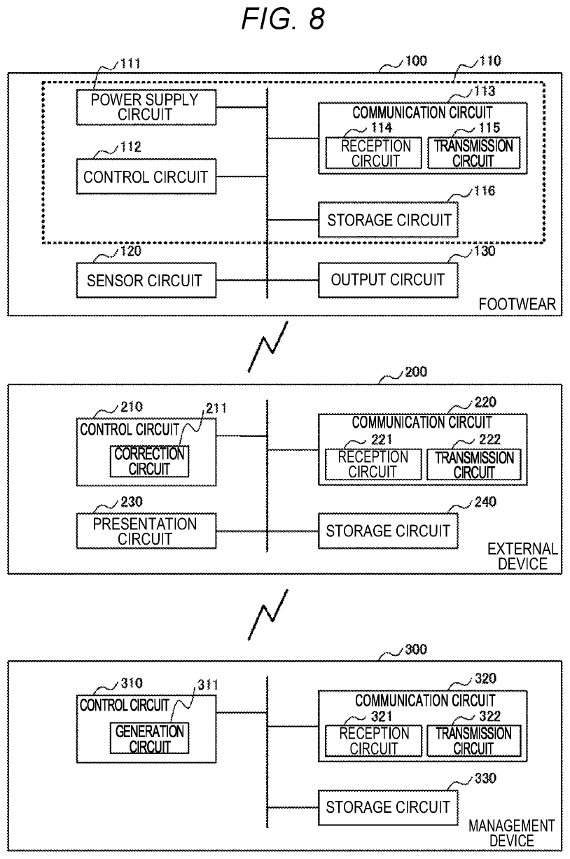

(10) Further, in the above embodiment, as a scheme for teaching walking in the device, the walking teaching is performed by a processor of the device executing a walking teaching program or the like, but this may be realized by a logic circuit (hardware) formed of an integrated circuit (an IC chip or large scale integration (LSI)) or the like or a dedicated circuit in the device. Further, these circuits may be realized by one or a plurality of integrated circuits, and functions of the plurality of functional units shown in the above embodiment may be realized by one integrated circuit. The LSI may be called a VLSI, a super LSI, an ultra LSI, or the like depending on a degree of integration. That is, as illustrated in FIG. 8, the footwear 100 may include a power supply circuit 111, a control circuit 112, a communication circuit 113, a storage circuit 116, a sensor circuit 120, and an output circuit 130, and functions of the respective circuits are the same as those of the respective units having the same names shown in the above embodiment. Similarly, the external device 200 may include a control circuit 210, a communication circuit 220, a presentation circuit 230, and a storage circuit 240. Further, the management device 300 may also include a control circuit 310, a communication circuit 320, and a storage circuit 330.

Further, the walking teaching program may be recorded on a processor-readable recording medium. A “non-transitory tangible medium” such as a tape, a disc, a card, a semiconductor memory, or a programmable logic circuit can be used as the recording medium. Further, the walking teaching program may be supplied to the processor via an arbitrary transmission medium (a communication network, broadcast waves, or the like) capable of transmitting the walking teaching program. The present invention can also be realized in the form of a data signal embedded in carrier waves, in which the walking teaching program has been embodied by electronic transmission.

The above walking teaching program can be implemented using, for example, script languages such as ActionScript and JavaScript (registered trademark), object-oriented programming languages such as Objective-C and Java (registered trademark), or markup languages such as HTMLS.

(11) The configuration illustrated in the above embodiment and the configuration illustrated in each supplement may be combined appropriately. Further, for respective processing procedures, execution procedures may be interchanged or two processes may be executed in parallel as long as the same results are obtained.

### REFERENCE SIGNS LIST

100 Footwear
110 Module
111 Power supply unit
112 Control unit
113 Communication unit
114 Reception unit
115 Transmission unit
116 Storage unit
120 Sensor unit
130 Output unit
200 External device
210 Control unit
211 Correction unit
220 Communication unit
221 Reception unit
222 Transmission unit
230 Presentation unit
240 Storage unit
300 Management device
301 Upper portion
302 Sole portion
303 Tongue portion
310 Control unit
311 Generation unit
320 Communication unit
321 Reception unit
322 Transmission unit
330 Storage unit
401 Groove portion
402, 403, 404, 405 Rib
501 Dent

1. A walking teaching system including footwear and an external device,
wherein the footwear includes
a sensor unit configured to detect a motion of the footwear; and
a first transmission unit configured to transmit detection information detected by the sensor unit to the external device, and
the external device includes
a first reception unit configured to receive the detection information;
a first storage unit configured to store model information serving as a model of walking; and
a presentation unit configured to present a way to walk to a user wearing the footwear on the basis of the detection information and the model information.

2. The walking teaching system according to claim 1,
wherein the walking teaching system further includes a management device,
the external device further includes a second transmission unit configured to transmit the received detection information to the management device,
the management device includes
a second reception unit configured to receive the detection information;
a second storage unit configured to store the received detection information;
a generation unit configured to generate correction information for correcting the model information on the basis of a plurality of pieces of detection information stored in the storage unit; and
a third transmission unit configured to transmit the correction information to the management device,
the first reception unit further receives the correction information,
the external device further includes a correction unit configured to correct the model information on the basis of the correction information, and
the presentation unit presents the way to walk on the basis of the model information corrected by the correction unit.

3. The walking teaching system according to claim 2,
wherein the first storage unit further stores user information indicating a feature of a user using the footwear,
the first storage unit stores a plurality of pieces of model information associated with the feature of the user, and
the presentation unit presents the way to walk on the basis of the model information corresponding to the feature of the user indicated by the user information and the detection information.

4. The walking teaching system according to claim 3,
wherein the second transmission unit further transmits the user information to the management device,
the second reception unit receives the user information, and
the generation unit generates correction information according to the feature of the user indicated by the user information.

5. The walking teaching system according to claim 2,
wherein the external device further includes a second generation unit configured to generate a control signal related to control of output of at least one of light, sound, and vibration on the basis of the detection information,
the second transmission unit further transmits the control signal to the footwear, and
the footwear further includes
a third reception unit configured to receive the control signal; and
an output unit configured to perform output of at least one of light, sound, and vibration on the basis of the control signal.

6. A walking teaching method using a walking teaching system including footwear and an external device, the walking teaching method comprising:
a detection step of detecting a motion of the footwear by the footwear;
a transmission step of transmitting, by the footwear, detection information detected in the detection step to the external device;
a storage step of storing, by the external device, model information serving as a model of walking; and
a presentation step of presenting, by the external device, a way to walk to a user wearing the footwear on the basis of the detection information and the model information.
